(19) United States
(12) Patent Application Publication
(10) Pub. No.: US 2022/0319045 A1
(43) Pub. Date: Oct. 6, 2022

(54) System For Posture Detection Using The Camera Of A Hand-Held Device

(71) Applicant: MohammadSado Lulu, El paso, TX (US)

(72) Inventor: MohammadSado Lulu, El paso, TX (US)

(21) Appl. No.: 17/301,404

(22) Filed: Apr. 1, 2021

(51) Int. Cl. G06T 7/73; G06T 3/40; G08B 21/18

(52) U.S. Cl. CPC G06T 7/74; G06T 3/4038; G08B 21/18; G06T 2207/30201

(57) ABSTRACT

A system for posture detection using a hand-held device such as a smartphone is disclosed. The disclosed is comprised of a memory, a processor, a camera device for measuring the angle at which the device is held, and a computer program product configured on the memory of the device and executed by its processor, such a product being capable of determining the posture of the holder of a device from the measured device angle. The system may further generate an alert to remind the user to adopt a proper posture. In essence, the built-in camera determines tracks the eyes and facial angle to determine if they are maintaining an appropriate angle when using the phone, and if not the phone would alert user.

### RELATED APPLICATIONS

U.S. Pat. No. 7,661,200B2: A sensor worn by a person measures movements of the person. Movements of orientation are not expressed by rotations leading from a stationary reference point to a reference point associated with the sensor, but by angles separating axes or planes of the stationary reference point and axes associated with the wearer, thereby making it possible to better identify his/her movements and his/her activity. The sensor can be applied to the monitoring of patients.

US20070032748A1: A portable sensor system that uses acceleration-insensitive, three-dimensional angle sensors located at various points on the patient's body, and collects data on the frequency and nature of the movements over extended periods of time..

U.S. Pat. No. 9,804,189B2: An upper body motion measurement system 1 has a plurality of inertia sensor units 2, each of which incorporating an angular velocity sensor 4 and an acceleration sensor 5. The plurality of the inertia sensor units 2 is attached to places that are different from each other on the upper body of a subject P. Based on the detection outputs of the angular velocity sensor 4 and the acceleration sensor 5, the attitude of each of the inertia sensor units 2 is estimated, and the acceleration thereof is further estimated. The angular acceleration of the upper body of the subject P is estimated based on the estimated accelerations of the plurality of the inertia sensor units 2.

### FIELD OF INVENTION

In general, the present invention relates to the use of posture measuring devices in health-care systems. More specifically, the invention relates to a system for posture detection using the camera of a hand-held device.

### BACKGROUND OF THE INVENTION

Sending short messages, emailing, scheduling appointments and browsing the internet on smartphones are some of the things such devices are used for. Smartphones are also used for teaching, work, meeting and learning activities in academic institutions. As a result of the increased usage of their smartphones, many people commonly report pain in the neck, shoulders and at the base of the thumb, which can be attributed to the postures they assume during smartphone usage. Over time maintaining unideal postures while using a smartphone may lead to physiological and biomechanical changes in body structures and the musculoskeletal system.

According to research, performing small repetitive motions (such as texting) associated with using smartphones in a sustained unideal posture during these motions, reduces circulation to muscle tissue. A reduction in circulation will limit sufficient nutrient supply to the muscles, which will result in the experience of localized pain and fatigue. If localized fatigue persists for more than 30 minutes, and there is a decrease in the maximum voluntary contractions of the muscle tissues, the effectiveness of the corticospinal-output may decrease. The reduction in corticospinal-output may result in reduced motor-neuron activity and will affect the control of movement.

Furthermore, postural changes, along with the associated local and global musculoskeletal problems that have been cited in the literature, are causes for concern because the young age of the majority of smartphone users can lead to sustained unideal postures into adulthood. If the extent of the problem is not explored and addressed, they may develop postural syndromes and musculoskeletal problems which might hinder the extent to which they can perform their professions and participate in recreational- and leisure activities.

This disclosure presents an approach to posture recognition using the camera sensor in hand-held devices. The aim is to use measurements from the camera for posture recognition, and to perform the method precisely enough to come up with a remedy to the sustained posture that may lead to adverse health effects.

### SUMMARY OF THE INVENTION

The following summary is an explanation of some of the general inventive steps for the system, method, architecture and tools in the description. This summary is not an extensive overview of the invention and does not intend to limit the scope beyond what is described and claimed as a summary.

In a first step, the technique involves a methodology of collecting data that is comprised of:

1. Starting from known postures to determine the approximate facial angle of the user at a user of a device faces its camera;

2. Determining the mathematical correlation between facial angle of the user and the posture of the holder from the previous process, and;

3. Determining a range of good posture i.e. between X and Y: X≤θ≤Y.

In the second step, the technique involves a methodology of determining posture from the measured facial angle that is comprised of:

1. Tracking the eyes and facial angle of the holder using a camera facing the user;

2. Using the correlation established in the claim 2 above to determine the posture of the holder, and;

3. Generating an alert if the posture is not within the established good posture range X≤θ≥Y.

### BRIEF DESCRIPTION OF THE FIGURES

FIG. 1 of the diagrams illustrates a person in a known posture θ and a known facial angle λ at which they hold the hand-held device, wherein it is established the correlation between posture and the facial angle at which a device is held.

FIG. 2 of the diagrams demonstrates a methodology and algorithm for estimating the posture of a person holding the device using the measured facial angle and its correlation with posture as determined using standard posture data.

FIG. 3 of the diagrams teaches of the practical application of the methodology of determining the posture angle β of a person holding a hand-held device such as a smartphone by measuring the facial angle δ.

FIG. 4 of the diagrams is a portable computing device configured with a computer program product, such a configuration allowing the device to generate alerts whenever bad posture is detected.

FIG. 5 of the diagrams illustrates the process of establishing the relationship between posture and facial angle at which a device is held with the goal of determining a range facial angle for which good posture is established.

FIG. 6 of the diagrams is the process of determining the posture of the holder of a hand-held device from a measured facial angle.

FIG. 7 of the diagrams is illustrated a person holding a hand-held device with a front-facing camera.

FIG. 8 of the diagrams illustrates a method of generating visual mimics to promote posture correction.

### DETAILED DESCRIPTION OF THE INVENTION

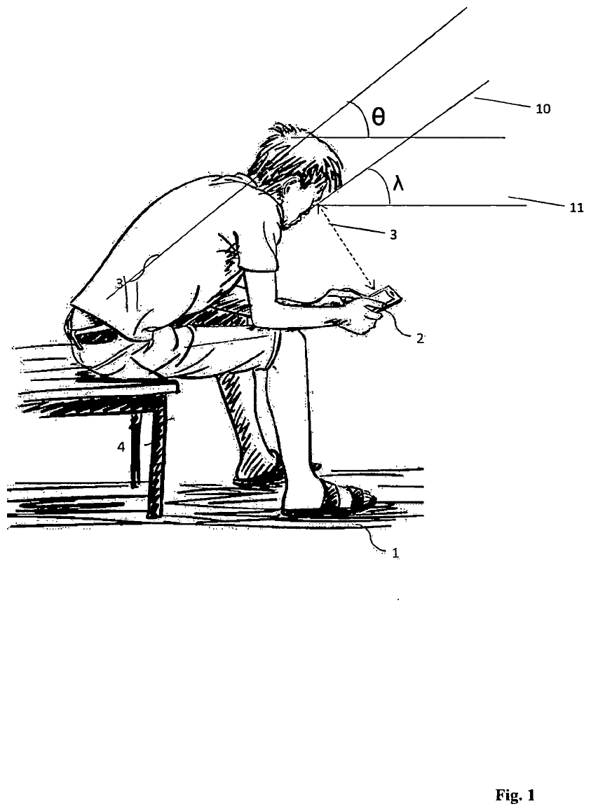

In the first embodiment according to FIG. 1 of the diagrams, it is illustrated a person 1 in a known posture 10, which is an angle θ and a known facial angle 11 labeled λ at which they hold the hand-held device 2, wherein it is established the correlation between posture and the facial angle λ at which a device is held. The diagram illustrates a line-of-sight 3, which is a straight line from the eye of the holder to a camera on the hand-held device. The line of sight is important to the process as it helps estimate the correlation between the facial angle λ and the posture θ when the test is carried out at different postures and consequently facial angles. The assumption for the methodology is that a user will maintain a predictable line of sight at any posture to be able to see the device properly and use it as necessary, thereby allowing a person skilled in the art to determine a correlation. Once a correlation is established, a range of good posture is determined i.e. good posture θ is between X and Y: X≤θ≤Y.

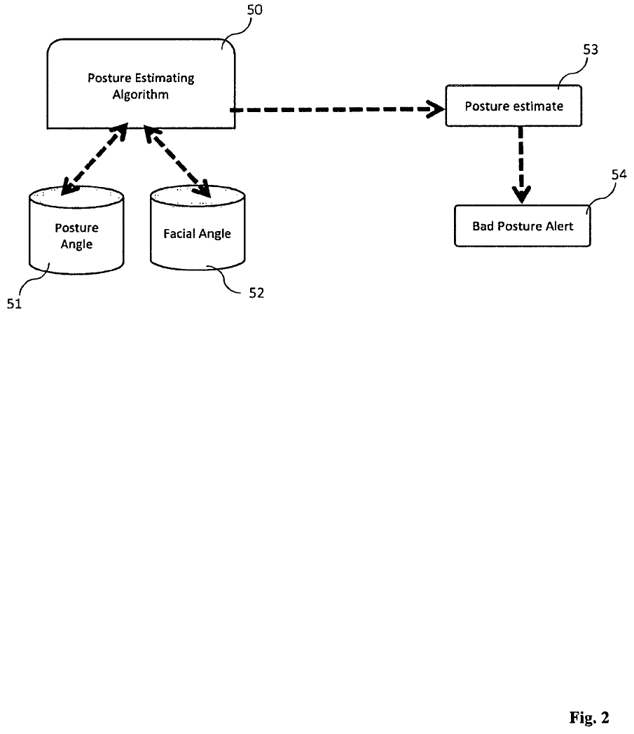

In the another embodiment according to FIG. 2 of the diagrams, it is demonstrated a methodology performed by a posture estimating algorithm 50 for estimating the posture of a person holding the device using the measured facial angle and its correlation with posture as determined using standard posture data. From FIG. 1 of the diagrams, it was possible to estimate the correlation between the facial angle λ and the posture θ when the test is carried out at different postures and consequently facial angles. That correlation is utilized by the posture estimating algorithm 50 in the following manner: the facial angle 52 is determined by a camera installed in the hand-held device,which tracks the eyes and facial angle; next, using the correlation between posture angle and facial angle of the algorithm, the facial angle 52 is weighed against posture angles in 51 to determine a posture estimate 53. Finally, wherein the posture estimate 53 is not within a predetermined range of good posture, a bad posture alert 54 is generated to alert the user.

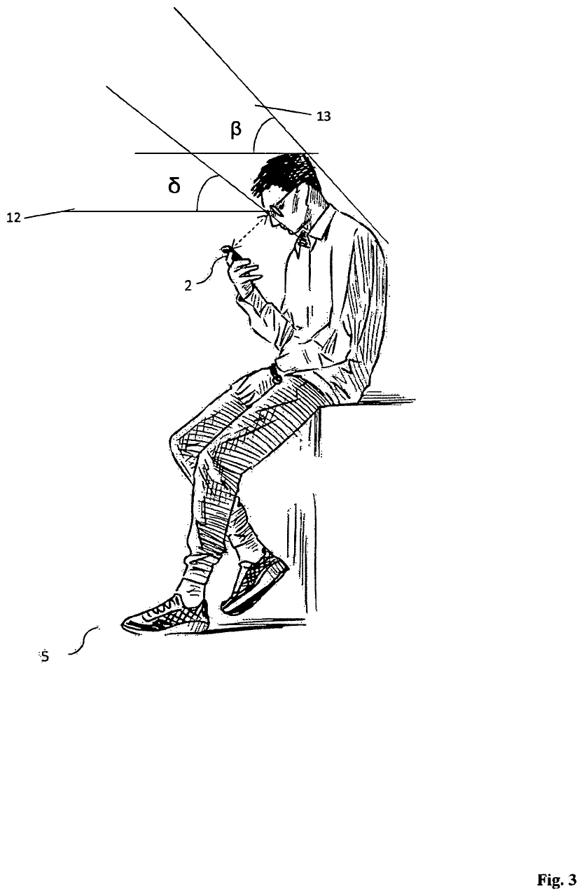

FIG. 3 of the diagrams is another embodiment that teaches of the practical application of the methodology of determining the posture angle β of a person holding a hand-held device such as a smartphone by measuring the facial angle δ. A person 5 using a hand-held 2 is shown. The angle δ, which the relative angle between the person's face and the device 2 is labeled 12 and is measured using a camera facing the user's/holder's face. The posture angle 13 of the holder is unknown, but can be determined as β using the system methodology that has been demonstrated in the description of FIG. 2 above.

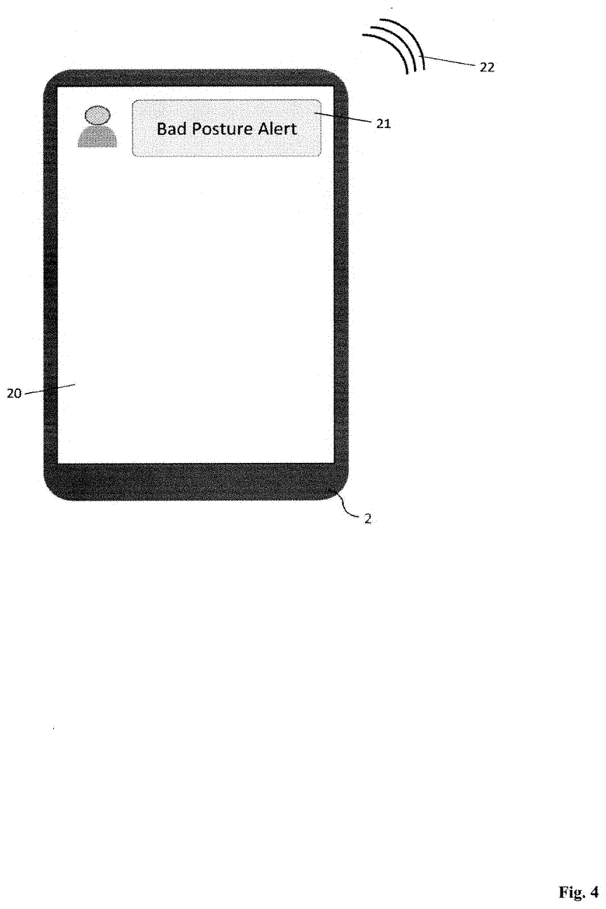

In another embodiment according to FIG. 4 of the diagrams, a hand-held device 2 is shown.

The device has a graphical user interface 20 and is configured with a computer program product, such a configuration allowing the device to generate a visual alert such as 21 or a sound or vibration alert such as 22 whenever bad posture is detected using the method and algorithm demonstrated by FIG. 2.

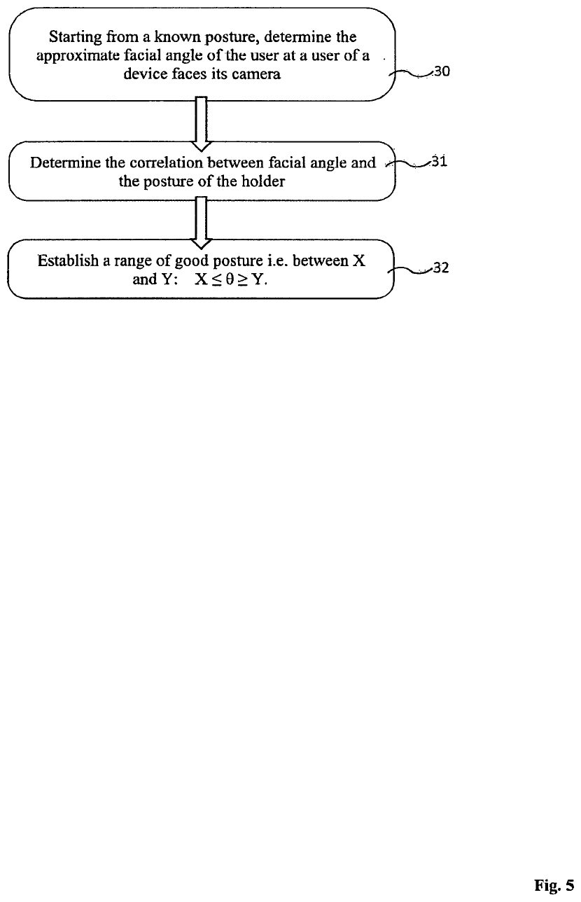

FIG. 5 of the diagrams illustrates the process of establishing the relationship between posture and the relative angle between the person's face and the device, with the goal of determining a range of facial angles for which good posture is established. In a process 30, the method involves starting from several known postures and determine the relative angle between the person's face and the device (facial angle) for each posture. In the process 31, it is determined the correlation between facial angle and the posture of the holder from the previous process 30. In a final step, it is determined a range of good postures i.e. between X and Y: X≤θ≥Y, to be used by the algorithm as in FIG. 2.

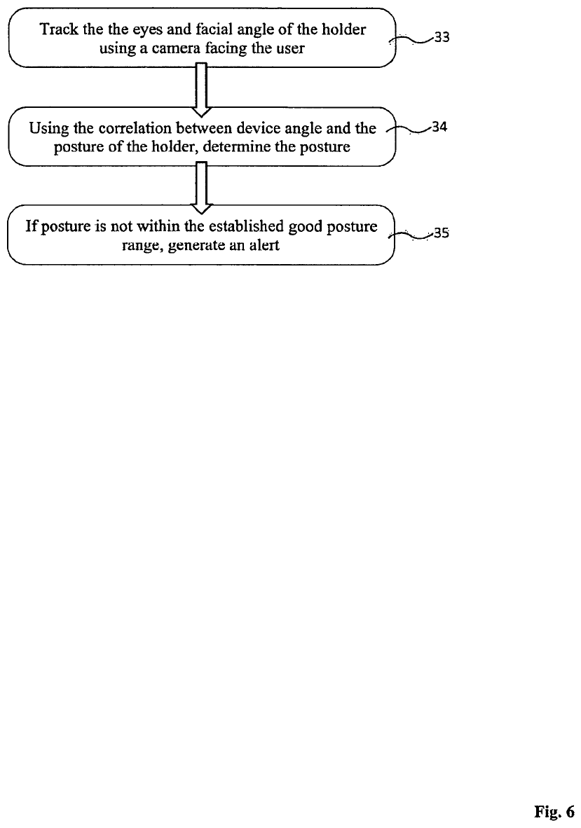

In another embodiment according to FIG. 6 of the diagrams is the process of determining the posture of the holder of a hand-held device from a measured facial angle. The method is characterized by tracking the eyes and facial angle of the holder using a camera facing the user in a process 33, using the correlation between the facial angle and the posture of the holder, determine the posture in a process 34 and generating an alert if the posture is not within the established good posture range X≤θ≤Y in a process 35.

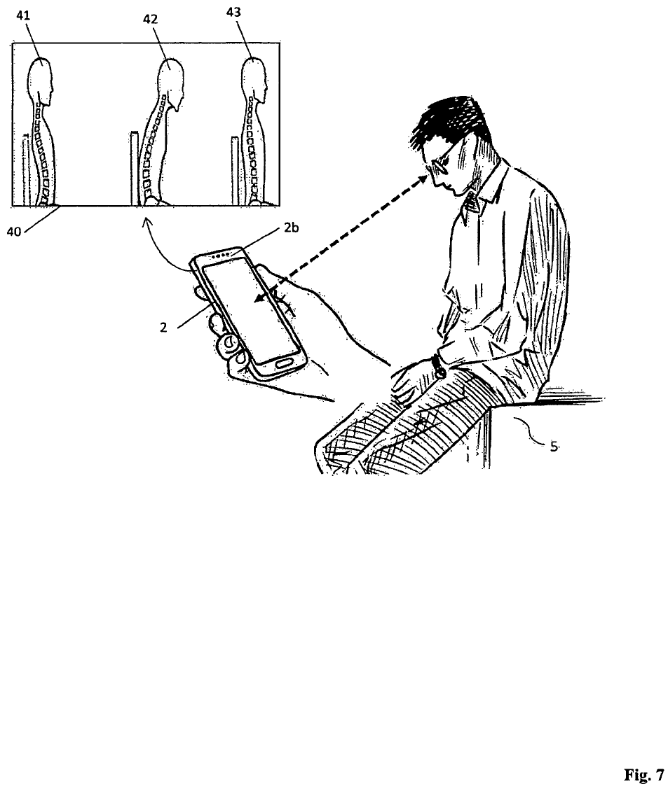

In another embodiment according to FIG. 7 of the diagrams is illustrated a person 5 holding a hand-held device 2. The device 2 has a front-facing camera 2b characterized in that the camera can take snapshots of the user when the user is facing the device while holding it in their hand. When the posture of a user of a device is detected as illustrated in the FIG. 6 above, the detected posture may further be correlated with the snapshots taken by the front-facing camera of the device. Preferably, the posture detected may be further characterized by generating a stream of visual mimics of user postures, and storing them in a storage device, each visual mimic correlating to a posture angle. Subsequently, the stored visual mimics may be related with snapshots taken by the front-facing camera, and the detected posture to generate visual mimics of the posture of the device holder illustrated as 40 in the figure. In the figure, 41, 42 and 43 illustrate different postures of the user in still images or as a stream of images stitched into video.

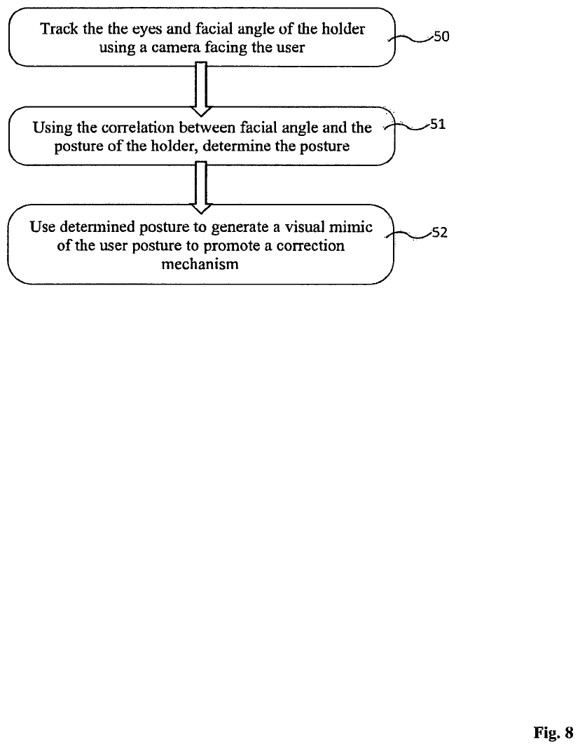

In another embodiment according to FIG. 8 of the diagrams is illustrated the method of generating visual mimics. In step 50, it illustrates the tracking the eyes and facial angle of the holder using a camera facing the user. The step 51 entails using the correlation established in the FIG. 6 above to determine the posture of the holder. Finally, 52 comprises of using the determined posture to generate a visual mimic of the user posture to illustrate the posture of device holder on its graphical interface. The process may further entail generating a visual alert on device to encourage good posture on the side of the user, such as 40 of FIG. 7 above. Moreover, the generation of visual mimic may be characterized of dynamic posture mimicking characterized in that a posture position is generated continuously for each gyroscope or accelerometer reading of the facial angle to create a stream of visual images stitched into a video.

### INDUSTRIAL APPLICATION

The current invention is applicable in the computing industry, specifically to manufacturing of hand-held portable computing devices.

What is claimed is:

1. A system for posture detection using a hand-held device such as a smartphone comprised of:
a memory;
a processor;
a camera facing the user of a phone capable of tracking the eyes and facial angle of the user, and;
a computer program product configured on the memory of the device and executed by its processor, such a product being capable of determining the posture of the holder of a device from the measured facial angle of the user.

2. The system for posture detection as in claim 1, wherein the method of establishing the relationship between posture and the angle of holding a device comprises of the steps of:
starting from known postures to determine the approximate facial angle of the user at a user of a device faces its camera;
determining the mathematical correlation between facial angle of the user and the posture of the holder from the previous process, and;
determining a range of good posture i.e. between X and Y: X≤θ≤Y.

3. The system for posture detection as in claim 1, wherein the method of determining the posture of the holder of a hand-held device from a measured device angle is comprised of the steps of:
tracking the eyes and facial angle of the holder using a camera facing the user;
using the correlation established in the claim 2 above to determine the posture of the holder, and;
generating an alert if the posture is not within the established good posture range X≤Θ≥Y.

4. The system for posture detection as in claim 3, wherein the generated alert is a visual alert such as 21 or a sound or vibration alert such as 22.

5. A method rendering a visual mimic of detected posture on the graphical user interface of a hand-held device comprising of the steps of:
tracking the eyes and facial angle of the holder using a camera facing the user;
using the correlation established in the claim 2 above to determine the posture of the holder, and;
using the determined posture to generate a visual mimic of the user posture to illustrate the posture of device holder on its graphical interface

6. The method as in claim 5, further comprising of generating a visual alert on device to encourage good posture

7. The method according to claim 5, wherein the generation of visual mimic is comprised of a dynamic posture mimicking characterized in that a posture position is generated continuously for each facial angle reading established to create a stream of visual images stitched into a video

8. A method of detecting the posture of the holder of a hand-held device characterized by:
generating a stream of visual mimics of user postures as in claim 5 above, and storing them in a storage device, each visual mimic correlating to a posture angle;
using the user-facing camera, capturing a picture or stream of pictures of the holder, and;
for each picture, comparing against stored visual mimics and using a mathematical transform to estimate the posture of the holder.
